(19) United States
(12) Patent Application Publication
(10) Pub. No.: US 2015/0087683 A1
(43) Pub. Date: Mar. 26, 2015

(54) TREATMENT METHOD FOR STEROID RESPONSIVE DERMATOSES

(71) Applicant: GlaxoSmithKline Intellectual Property (No.2) Limited, Brentford, Middlesex (GB)

(72) Inventors: Pankaj Agarwal, King of Prussia, PA (US); Vinod Kumar, King of Prussia, PA (US); Qing Xie, King of Prussia, PA (US)

(21) Appl. No.: 14/394,394

(22) PCT Filed: Apr. 22, 2013
(86) PCT No.: PCT/EP2013/058249

§ 371 Date: Oct. 14, 2014

Related U.S. Application Data
(60) Provisional application No. 61/637,390, filed on Apr. 24, 2012.
(60) Provisional application No. 61/782,565, filed on Mar. 14, 2013.

(51) Int. Cl. A61K 31/4155; A61K 9/00

(52) U.S. Cl. CPC A61K 31/4155; A61K 9/0014
USPC 514/406; 548/365.7

(57) ABSTRACT

Invented is a method of treating steroid responsive dermatoses in a mammal, including a human, in need thereof which comprises the administration of a therapeutically effective amount of a compound selected from the group consisting of: N-{(1S)-2-Amino-1-[(3-fluorophenyl)methyl]ethyl}-5-chloro-4-(4-chloro-1-methyl-1H-pyrazol-5-yl)-2-thiophenecarboxamide or a pharmaceutically acceptable salt thereof, and the compound N-{(1S)-2-amino-1-[(3,4-difluorophenyl)methyl]ethyl}-5-chloro-4-(4-chloro-1-methyl-1H-pyrazol-5-yl)-2-furancarboxamide or a pharmaceutically acceptable salt thereof, to such mammal.

### FIELD OF THE INVENTION

This invention relates to a method of treating steroid responsive dermatoses in a mammal, suitably a human, by administering a compound selected from the group consisting of: N-{(1S)-2-amino-1-[(3-fluorophenyl)methyl]ethyl}-5-chloro-4-(4-chloro-1-methyl-1H-pyrazol-5-yl)-2-thiophenecarboxamide or a pharmaceutically acceptable salt thereof, suitably the hydrochloride salt; and N-{(1S)-2-amino-1-[(3,4-difluorophenyl)methyl]ethyl}-5-chloro-4-(4-chloro-1-methyl-1H-pyrazol-5-yl)-2-furancarboxamide or a pharmaceutically acceptable salt thereof.

### BACKGROUND OF THE INVENTION

N-{(1S)-2-Amino-1-[(3-fluorophenyl)methyl]ethyl}-5-chloro-4-(4-chloro-1-methyl-1H-pyrazol-5-yl)-2-thiophenecarboxamide (hereinafter Compound A) is a compound which is disclosed and claimed, along with pharmaceutically acceptable salts thereof, as being useful in the treatment of cancer and arthritis, in International Application No. PCT/US08/053,269, having an International filing date of Feb. 7, 2008; International Publication Number WO 08/098,104 and an International Publication date of Aug. 14, 2008 (compound of Example 96). The entire disclosure of which is hereby incorporated by reference.

The crystalline hydrochloride salt of Compound A: N-{(1S)-2-Amino-1-[(3-fluorophenyl)methyl]ethyl}-5-chloro-4-(4-chloro-1-methyl-1H-pyrazol-5-yl)-2-thiophenecarboxamide hydrochloride (hereinafter Compound B) is a compound which is disclosed and claimed as being useful in the treatment of cancer and arthritis, in International Application No. PCT/US2010/022323, having an International filing date of Jan. 28, 2010; International Publication Number WO 2010/088331 and an International Publication date of Aug. 5, 2010. The entire disclosure of which is hereby incorporated by reference.

N-{(1S)-2-amino-1-[(3,4-difluorophenyl)methyl]ethyl}-5-chloro-4-(4-chloro-1-methyl-1H-pyrazol-5-yl)-2-furancarboxamide, or a pharmaceutically acceptable salt thereof (hereinafter Compound C) is a compound which is disclosed and claimed as being useful in the treatment of cancer and arthritis, in International Application No. PCT/US08/053,269, having an International filing date of Feb. 7, 2008; International Publication Number WO 08/098,104 and an International Publication date of Aug. 14, 2008 (compound of Example 224).

Suitably, the present invention concerns novel therapeutic uses of Compound A or a pharmaceutically acceptable salt thereof, suitably novel therapeutic uses of Compound B, suitably novel therapeutic uses of Compound C.

### SUMMARY OF THE INVENTION

This invention relates to a method of treating steroid responsive dermatoses in a mammal, including a human, in need thereof which comprises administering to such mammal a therapeutically effective amount of the compound selected from the group consisting of: N-{(1S)-2-amino-1-[(3-fluorophenyl)methyl]ethyl}-5-chloro-4-(4-chloro-1-methyl-1H-pyrazol-5-yl)-2-thiophenecarboxamide or a pharmaceutically acceptable salt thereof, and N-{(1S)-2-amino-1-[(3,4-difluorophenyl)methyl]ethyl}-5-chloro-4-(4-chloro-1-methyl-1H-pyrazol-5-yl)-2-furancarboxamide or a pharmaceutically acceptable salt thereof.

This invention also relates to a method of treating steroid responsive dermatoses in a mammal, including a human, in need thereof which comprises administering to such mammal a therapeutically effective amount of the compound N-{(1S)-2-amino-1-[(3-fluorophenyl)methyl]ethyl}-5-chloro-4-(4-chloro-1-methyl-1H-pyrazol-5-yl)-2-thiophenecarboxamide hydrochloride wherein the compound is administered topically.

This invention also relates to a method of treating steroid responsive dermatoses in a mammal, including a human, in need thereof which comprises administering to such mammal a therapeutically effective amount of the compound N-{(1S)-2-amino-1-[(3-fluorophenyl)methyl]ethyl}-5-chloro-4-(4-chloro-1-methyl-1H-pyrazol-5-yl)-2-thiophenecarboxamide or a pharmaceutically acceptable salt thereof wherein the compound is administered topically.

This invention also relates to a method of treating steroid responsive dermatoses in a mammal, including a human, in need thereof which comprises administering to such mammal a therapeutically effective amount of the compound N-{(1S)-2-amino-1-[(3-fluorophenyl)methyl]ethyl}-5-chloro-4-(4-chloro-1-methyl-1H-pyrazol-5-yl)-2-thiophenecarboxamide wherein the compound is administered topically.

This invention also relates to a method of treating steroid responsive dermatoses in a mammal, including a human, in need thereof which comprises administering to such mammal a therapeutically effective amount of the compound N-{(1S)-2-amino-1-[(3,4-difluorophenyl)methyl]ethyl}-5-chloro-4-(4-chloro-1-methyl-1H-pyrazol-5-yl)-2-furancarboxamide or a pharmaceutically acceptable salt thereof wherein the compound is administered topically.

This invention also relates to a method of treating steroid responsive dermatoses in a mammal, including a human, in need thereof which comprises administering to such mammal a therapeutically effective amount of the compound N-{(1S)-2-amino-1-[(3,4-difluorophenyl)methyl]ethyl}-5-chloro-4-(4-chloro-1-methyl-1H-pyrazol-5-yl)-2-furancarboxamide wherein the compound is administered topically.

This invention also relates to a method of treating steroid responsive dermatoses in a mammal, including a human, in need thereof which comprises administering to such mammal a therapeutically effective amount of the compound N-{(1S)-2-amino-1-[(3-fluorophenyl)methyl]ethyl}-5-chloro-4-(4-chloro-1-methyl-1H-pyrazol-5-yl)-2-thiophenecarboxamide or a pharmaceutically acceptable salt thereof wherein the compound is administered orally.

This invention also relates to a method of treating steroid responsive dermatoses in a mammal, including a human, in need thereof which comprises administering to such mammal a therapeutically effective amount of the compound N-{(1S)-2-amino-1-[(3,4-difluorophenyl)methyl]ethyl}-5-chloro-4-(4-chloro-1-methyl-1H-pyrazol-5-yl)-2-furancarboxamide or a pharmaceutically acceptable salt thereof wherein the compound is administered orally.

This invention also relates to a method of treating steroid responsive dermatoses in a mammal, including a human, in need thereof which comprises administering to such mammal a therapeutically effective amount of the compound N-{(1S)-2-amino-1-[(3-fluorophenyl)methyl]ethyl}-5-chloro-4-(4-chloro-1-methyl-1H-pyrazol-5-yl)-2-thiophenecarboxamide or a pharmaceutically acceptable salt thereof wherein the compound is administered parenterally.

This invention also relates to a method of treating steroid responsive dermatoses in a mammal, including a human, in need thereof which comprises administering to such mammal a therapeutically effective amount of the compound N-{(1S)-2-amino-1-[(3,4-difluorophenyl)methyl]ethyl}-5-chloro-4-(4-chloro-1-methyl-1H-pyrazol-5-yl)-2-furancarboxamide or a pharmaceutically acceptable salt thereof wherein the compound is administered parenterally.

Included in the present invention are pharmaceutical compositions comprising a pharmaceutical carrier and compounds useful in the methods of the invention.

Also included in the present invention are methods of co-administering compounds useful in the methods of the invention with further active ingredients.

### DETAILED DESCRIPTION OF THE INVENTION

This invention relates to methods of treating steroid responsive dermatoses in a mammal, including a human, in need thereof which comprises administering to such mammal a therapeutically effective amount of the compound selected from the group consisting of: N-{(1S)-2-amino-1-[(3-fluorophenyl)methyl]ethyl}-5-chloro-4-(4-chloro-1-methyl-1H-pyrazol-5-yl)-2-thiophenecarboxamide (Compound A) or a pharmaceutically acceptable salt thereof, and N-{(1S)-2-amino-1-[(3,4-difluorophenyl)methyl]ethyl}-5-chloro-4-(4-chloro-1-methyl-1H-pyrazol-5-yl)-2-furancarboxamide or a pharmaceutically acceptable salt thereof. Suitably the pharmaceutically acceptable salt of Compound A is the hydrochloride salt or N-{(1S)-2-amino-1-[(3-fluorophenyl)methyl]ethyl}-5-chloro-4-(4-chloro-1-methyl-1H-pyrazol-5-yl)-2-thiophenecarboxamide hydrochloride (Compound B).

Steroid responsive dermatoses are those dermatological disorders that will respond to topical corticosteroid treatment. Specific steroid responsive dermatoses may be treated according to the presently invented methods. Suitably, the invention relates to a method of treating steroid responsive dermatoses in a mammal, including a human, wherein the dermatoses is selected from: contact dermatitis, eczema, infantile eczema, atopic dermatitis, ichthyosis, psoriasis, xeroderma, seborrheic dermatitis, nummular dermatitis, dermatitis herpetiformis, neurodermatitis, stasis dermatitis, lichen simplex chronicus, dermatophytids, candidiasis, intertrigo, scabies, pityriasis rosea, lichen planus, pityriasis rubra pilaris, bullous pemphigoid, miliaria, acute and chronic eczema, lupus erythematosis, photoallergic reactions, pruritis, and combinations thereof.

Suitably, the dermatoses is selected form: contact dermatitis, eczema, infantile eczema, atopic dermatitis, psoriasis, seborrheic dermatitis, nummular dermatitis, dermatitis herpetiformis, neurodermatitis, stasis dermatitis, dermatophytids, acute eczema and chronic eczema.

Suitably, the dermatoses is selected form: eczema, atopic dermatitis and psoriasis.

Suitably, the dermatoses is: psoriasis.

The steroid responsive dermatoses treated according to the presently invented methods can have a variety of causes. Several non-limiting examples of such causes include hypersensitivity, IgE mediation, anti-membrane antibody, immune complex disease, cell mediated immunity, and combinations thereof.

The steroid responsive dermatoses may also be caused by an insult to a tissue of the mammal having the dermatoses. Several non-limiting examples of such insults include a physical insult, a chemical insult, an environmental insult, a topically mediated insult, an internally mediated insult, and combinations thereof.

The steroid responsive dermatoses may be a secondary physiologic response to a primary disease. Several non-limiting examples of such primary diseases causative of the steroid responsive dermatoses include an infection, an allergic response, a hyperproliferative disorder, an immunologic disorder, a metabolic disorder, a drug induced response, a disorder related to proper or improper organ function, and combinations thereof.

The methods and compositions of the invention are useful in treating steroid responsive dermatoses regardless of the cause of the disease.

Psoriasis is a T cell mediated disorder that is characterized by the presence of memory effector T cells (CD45RO+), and increased proliferation and reduced differentiation of keratinocytes in skin lesions (Schon and Boehncke. N Engl J. Med. 2005). Suitably, the invention relates to a method of treating psoriasis.

By the term “treating” and derivatives thereof as used herein, is meant prophylatic and therapeutic therapy. Prophylactic therapy is appropriate, for example, when a subject is considered at high risk for developing a steroid responsive dermatoses, such as when a subject has a strong family history of steroid responsive dermatological disorders, or when a subject has a history of repeating or seasonal steroid responsive dermatological disorders.

Prophylactic use of a compound of this invention is contemplated whenever numerous causative factors are present in a subject. Prophylactic uses of the methods of this invention include but are not limited to treatment of a subject with a history of repeating or seasonal steroid responsive dermatological disorders before the disease is detectable.

By the phrases “to a therapeutic extent”, “treating” and “therapeutically effective amount” and derivatives thereof as used herein, unless otherwise defined, is meant that amount of Compound A or a pharmaceutically acceptable salt thereof, or Compound B, or Compound C, that will elicit the biological or medical response of a tissue, system, mammal or human that is being sought, for instance, by a researcher or clinician. Furthermore, the term “therapeutically effective amount” means any amount which, as compared to a corresponding subject who has not received such amount, results in improved treatment, healing, prevention, lessening in severity or amelioration of steroid responsive dermatoses.

As indicated above, steroid responsive dermatoses is known to have many causative factors. This invention relates to the treatment of steroid responsive dermatoses regardless of the factor or factors causing the condition. The pharmaceutically active compounds of this invention are also useful in treating steroid responsive dermatoses, suitably psoriasis, when the causative factor or factors of the condition are unknown or have yet to be identified.

A skilled physician will be able to determine the appropriate situation in which subjects are affected by or susceptible to steroid responsive dermatoses, suitably psoriasis, for administration by methods of the present invention.

Certain of the compounds described herein may contain one or more chiral atoms, or may otherwise be capable of existing as two enantiomers. Accordingly, the compounds of this invention include mixtures of enantiomers as well as purified enantiomers or enantiomerically enriched mixtures. Also, it is understood that all tautomers and mixtures of tautomers are included within the scope of the compounds of the invention.

Certain compounds described herein may form a solvate which is understood to be a complex of variable stoichiometry formed by a solute (for example, Compound A or a pharmaceutically acceptable salt thereof) and a solvent. Such solvents for the purpose of the invention may not interfere with the biological activity of the solute. Examples of suitable solvents include, but are not limited to, water, methanol, ethanol and acetic acid. Preferably, the solvent used is a pharmaceutically acceptable solvent. Examples of suitable pharmaceutically acceptable solvents include, without limitation, water, ethanol and acetic acid. Suitably the solvent used is water.

The pharmaceutically acceptable salts of the compounds of the invention are readily prepared by those of skill in the art.

The treatment of steroid responsive dermatoses, as described herein, is accomplished by the administration of Compound A or a pharmaceutically acceptable salt thereof, or Compound B, or Compound C, and is not limited to any particular mechanism of action.

When referring to the treatment of steroid responsive dermatoses, the term “co-administering” and derivatives thereof as used herein is meant either simultaneous administration or any manner of separate sequential administration of Compound A or a pharmaceutically acceptable salt thereof, or Compound B, or Compound C and a further active ingredient or ingredients, known to be useful in the treatment of steroid responsive dermatoses. The term further active ingredient or ingredients, as used herein, includes any compound or therapeutic agent known to or that demonstrates advantageous properties when administered to a patient in need of treatment for steroid responsive dermatoses. Preferably, if the administration is not simultaneous, the compounds are administered in a close time proximity to each other. Furthermore, it does not matter if the compounds are administered in the same dosage form, e.g. one compound may be administered topically and another compound may be administered orally.

Typically, any agent that has activity versus a steroid responsive dermatoses being treated may be co-administered in the treatment of steroid responsive dermatoses in the present invention.

The current invention relates to the use of Compound A or a pharmaceutically acceptable salt thereof, Compound B, and Compound C in the treatment of steroid responsive dermatoses in mammals, including humans.

The ability of the compounds of the invention to treat steroid responsive dermatoses is demonstrated by activity in the following Assay.

### Assay #1

A high-throughput screen was utilized to compare mRNA expression patterns in psoriatic human skin (clinical samples) and normal human skin (clinical samples) and associate the differences with the effects of pharmaceutically active compounds. The screen generally followed the methodology in Lamb et al., Science 313, 1929 (2006).

A general overview of the experimental protocol is indicated below.

### General Experimental Protocol

Seed cells: Day 1

Cells are grown in culture for about 24 hours to prepare a T-225 flask of MCF7s that are ready to be seeded for the assay.
Growth media is aspirated from cells.
The cell monolayer is rinsed once with 10-15 mL DPBS, then aspirated.
2 ml 0.05% Trypsin-EDTA is added and the flask is tilted to cover the bottom. The flask is incubated at 37 C for ˜2 minutes to dissociate the cells.
The dissociated cells are harvested with culture media, ˜10 ml/flask.
The harvested cell suspensions are pooled and re-suspended to ensure a homogenous suspension.
The suspension is divided into approximately 1e10̂6 cells per well.
The cells are spun down and re-suspended in fresh culture media to 1e10̂6 cells/2 mL media.
2 ml of the adjusted cell suspension is dispensed into each well of 6-well plates.
The 6-well plates are incubated overnight at approximately 37 C: 5% CO2.

### Compound Treatment: Day 2 (AM)

An assay block, containing 3 ul 10 mM compound (small molecule)/well, is allowed to return to room temperature.
Culture media is warmed to 37 C.
600 ul culture media is added to each well of assay block (containing 3 ul 10 mM compound). (=200× dilution)
Intermediate dilution is then 50 uM.
500 ul of the intermediate dilution is transferred directly on to cells in 6-wells plated (in 2 ml) from previous day. (=5× dilution)
Cells then have 2.5 ml media on them. Final compound concentration is then 10 uM, final DMSO concentration is then 0.1%.
Treated cells are then returned to the incubator and treatment time is noted.
By way of example, if the final treatment concentration is to be something other than 10 uM, the stock should be adjusted first in DMSO so that the 1000× dilution still delivers 0.1% DMSO final. For example, a compound to be tested at 1 uM final should have a 1 mM stock in 100% DMSO prepared, then 3 ul into 600 ul media and 500 ul onto 2 mL steps will be the same.
Alternatively, a peptide or non-DMSO stock compound should be diluted in 600 ul media to which 3 ul of DMSO has been dissolved.

### Sample Harvest: Day 2 (PM)

Fresh RLT buffer (QIAGEN)+2-Me is prepared.
After 6 hours of treatment, the 6-well plates are removed from incubator.
The test compound is aspirated from the cell monolayer.
350 ul of RLT buffer is added per well.
Cells are scraped and the contents of each well is transferred to an appropriately labeled microfuge tube.
Freeze and store the samples at −80 C.

### RNA Profiling and Data Generation

Frozen samples were shipped to Expression Analysis, Inc. Durham N.C. (EA) to generate RNA profiling data. To generate whole genome RNA expression profiles, samples were processed for RNA purification and hybridization to IllumanHT-12-V4 Expression BeadChips. RNA profiling raw data generated from EA was further analyzed to produce a list of genes ranked by compound induced mRNA expression changes.

In general, the analysis of the obtained samples consists of generating an enrichment score between the query disease signature and each compound-specific expression profile using a nonparametric, rank-based pattern-matching based on the Kolmogorov-Smirnov statistic Subramanian et al. Proc. Natl. Acad. Sci. Vol. 102 no. 43 15,545-15550 (2005). The significance of particular set of compound instance(s) of interest is estimated by permutation pvalue. The results of Assay 1 for Compounds C and B are indicated in Table 1.

TABLE 1
Table 1. Results from CMap analysis using psoriasis disease signature against
compound profiles derived from from MCF7 cells treated with compounds C and B.

| | | | | Mean CMap | Enrichment | CMap Score | Compound | Cell |
|---|---|---|---|---|---|---|---|---|
| Compound | P-Value | Specificity | n | Score | Score | Distribution | Conc. | Type |
| C | 0 | 0.0104 | 6 | −0.1867 | −0.8835 | (−0.17 −0.17 | 10 uM | MCF7 |
| | | | | | | −0.17 −0.20 −0.20 | | |
| | | | | | | −0.21) | | |
| B | 0.013 | 0.2304 | 3 | −0.1633 | −0.8121 | (−0.13 −0.16 | 10 uM | MCF7 |
| | | | | | | −0.20) | | |

Based on the results in the above assay, Compounds B and C generated a set of gene expression changes which were negatively correlated with the set of gene expression changes observed in psoriasis, indicating a rational for its use in psoriasis and other steroid responsive dermatoses.

### Assay #2

The general procedure of Assay #1 was repeated with Compound C with substitution of primary keratinocytes. Primary keratinocytes were purchased from Zen-Bio, Inc. and grown in Zen-Bio, Inc. human adult keratinocyte growth medium (cat# KM-2).

Following the general procedure of Assay #1 for Day 1, keratinocytes plated on T225 flasks were trypsinized and resuspended in 17.4 mL of KM-2 medium. The cells were then seeded in the 6-well plates with 0.5×106 cells per well in a total 4 mL KM-2 medium.

Following the general procedure of Assay #1 for Day 2, keratinocytes were treated with the compound at the final concentrations of 1 uM and 10 uM for 6 hours. KM-2 medium containing test compound is aspirated from the cell monolayer. 350 ul of RLT buffer is added per well, the contents in each well were scraped and transferred to an appropriately labeled microfuge tube. Samples were frozen and stored at −80 C.

The subsequent sample processing, RNA purification and hybridization to the chips for microarray experiments were conducted by Expression Analysis, Inc. Durham N.C. (EA). Raw data generated from EA was further analyzed to generate a list of genes ranked by compound induced mRNA expression changes. The results of Assay 2 for Compound C are indicated in Table 2.

TABLE 2
Table 2. Results from CMap analysis using psoriasis disease signature
against compound profiles derived from Keratinocytes treated with compound C.

| | Raw | Scale | | | | Compound | |
|---|---|---|---|---|---|---|---|
| Compound | Score | Score | Ks up | Ks down | Day | Conc. | Cell Type |
| C | −0.332382 | −0.6723 | −0.22 | 0.112 | 1 | 1 uM | Keratinocyte |
| C | −0.420439 | −0.85041 | −0.248 | 0.172 | 2 | 1 uM | Keratinocyte |
| C | −0.262476 | −0.530904 | −0.139 | 0.124 | 3 | 1 uM | Keratinocyte |
| C | −0.325131 | −0.657634 | −0.213 | 0.112 | 1 | 10 uM | Keratinocyte |
| C | −0.41045 | −0.830207 | −0.296 | 0.115 | 2 | 10 uM | Keratinocyte |
| C | −0.355069 | −0.718188 | −0.241 | 0.114 | 3 | 10 uM | Keratinocyte |

Based on the results in the above Assay #2, Compound C generated a set of gene expression changes which were negatively correlated with the set of gene expression changes observed in psoriasis, indicating a rational for its use in psoriasis and other steroid responsive dermatoses.

### Clinical Result

Compound C was in human clinical trials for the treatment of cancer. In trial PCS113124, patient 1200 entered with mild plaques of psoriasis which generally got better and even disappeared at times during treatment with Compound C.

Moreover, assays useful for identifying compounds that exhibit therapeutic activity in psoriasis are well known to those of skill in the art. Such assays are also useful in confirming that Compound A or a pharmaceutically acceptable salt thereof, Compound B, and Compound C are useful in the treatment of psoriasis and other steroid responsive dermatoses.

Many current treatments for steroid responsive dermatoses, particularly steroidal treatments, are toxic to the patient and/or are known to trigger adverse events including, blurred vision, halos around lights, an irregular heartbeat, insomnia, mood changes, weight gain, fatigue, redness, blistering, burning, itching, peeling, thinning or the skin, and stretch marks. Such adverse events are particularly prevalent in children. One advantage of the compounds of the invention is that the compounds are non-steroidal compounds and are not associated with the adverse events of steroidal compounds. This advantage in the treatment of steroid responsive dermatoses can be realized whether Compound A or a pharmaceutically acceptable salt thereof, Compound B, or Compound C, is being administered alone or whether another steroid responsive dermatoses agent is being co-administered.

The present invention therefore provides a method of treating steroid responsive dermatoses in a mammal, including a human, including wherein the dermatological disorder is selected from the group consisting of: contact dermatitis, eczema, infantile eczema, atopic dermatitis, ichthyosis, psoriasis, xeroderma, seborrheic dermatitis, nummular dermatitis, dermatitis herpetiformis, neurodermatitis, stasis dermatitis, lichen simplex chronicus, dermatophytids, candidiasis, intertrigo, scabies, pityriasis rosea, lichen planus, pityriasis rubra pilaris, bullous pemphigoid, miliaria, acute and chronic eczema, lupus erythematosis, photoallergic reactions, pruritis, and combinations thereof, which comprises the administration an effective amount of Compound A or a pharmaceutically acceptable salt thereof, or Compound B, or Compound C, to a mammal, including a human, in need thereof.

In another embodiment of the invention the human is a pediatric patient.

The compounds of the invention may be administered to a patient in need thereof by any conventional route of administration, including, but not limited to, topical, intravenous, intramuscular, oral, subcutaneous, intradermal, and parenteral. Suitably, the compounds of the invention are administered topically. When used in a combination, each active compound can be administered alone or together. The compounds may be administered topically either in combination with each other, or sequentially in any order For example a first active compound may be administered, followed by a second active compound. In another embodiment of the invention, at least one active compound may be administered by a different route than the other compound(s). For example, a first active compound may be administered topically and a second active compound may be administered orally, etc.

The compounds of the present invention are incorporated into convenient dosage forms such as topical formulations, capsules, tablets, or injectable preparations. Solid or liquid pharmaceutical carriers are employed. Solid carriers include, starch, lactose, calcium sulfate dihydrate, terra alba, sucrose, talc, gelatin, agar, pectin, acacia, magnesium stearate, and stearic acid. Liquid carriers include syrup, peanut oil, olive oil, saline, and water. Similarly, the carrier or diluent may include any prolonged release material, such as glyceryl monostearate or glyceryl distearate, alone or with a wax. The amount of solid carrier varies widely but, preferably, will be from about 25 mg to about 1 g per dosage unit. When a liquid carrier is used, the preparation will suitably be in the form of a syrup, elixir, emulsion, soft gelatin capsule, sterile injectable liquid such as an ampoule, or an aqueous or nonaqueous liquid suspension.

The pharmaceutical preparations are made following conventional techniques of a pharmaceutical chemist involving mixing, granulating, and compressing, when necessary, for tablet forms, or mixing, filling and dissolving the ingredients, as appropriate, to give the desired oral or parenteral products.

Suitably, the compounds of the invention are administered topically. Pharmaceutical formulations adapted for topical administration may be formulated as ointments, creams, suspensions, lotions, powders, solutions, pastes, gels, sprays, aerosols or oils.

The amount of active ingredient required for therapeutic effect on topical administration will, of course vary with the compound chosen, the nature and severity of the disease state being treated and the mammal undergoing treatment, and is ultimately at the discretion of the physician. A suitable dose of each active ingredient may independently be from about 0.5 mg to 500 mg for topical administration, suitably from 1 mg to 100 mg, for example 2 to 25 mg administered from one to six times daily, suitably from one to three times daily.

By topical administration is meant non-systemic administration and includes the application of the active ingredient externally to the epidermis, to the buccal cavity and instillation of such a compound into the ear, eye and nose, and where the compound does not significantly enter the blood stream, suitably by application of the active ingredient externally to the epidermis. By systemic administration is meant oral, intravenous, intraperitoneal and intramuscular administration.

While it is possible for an active ingredient to be administered alone as the raw chemical, it is preferable to present it as a pharmaceutical formulation. Each active ingredient may comprise, for topical administration, from 0.001% to 10% w/w, e.g. from 1% to 2% by weight of the formulation although it may comprise as much as 10% w/w but preferably not in excess of 5% w/w and more preferably from 0.1% to 1% w/w of the formulation.

The topical formulations of the present invention, both for veterinary and for human medical use, comprise an active ingredient together with one or more acceptable carrier(s) and optionally any other therapeutic ingredient(s). The carrier(s) must be ‘acceptable’ in the sense of being compatible with the other ingredients of the formulation and not deleterious to the recipient thereof.

Formulations suitable for topical administration include liquid or semi-liquid preparations suitable for penetration through the skin to the site of inflammation such as liniments, lotions, creams, ointments or pastes.

Lotions according to the present invention include those suitable for application to the skin or eye. An eye lotion may comprise a sterile aqueous solution optionally containing a bactericide and may be prepared by methods similar to those for the preparation of drops. Lotions or liniments for application to the skin may also include an agent to hasten drying and to cool the skin, such as an alcohol or acetone, and/or a moisturizer such as glycerol or an oil such as castor oil or arachis oil.

Creams, ointments or pastes according to the present invention are semi-solid formulations of the active ingredient for external application. They may be made by mixing the active ingredient in finely divided or powdered form, alone or in solution or suspension in an aqueous or non-aqueous fluid, with the aid of suitable machinery, with a greasy or non-greasy basis. The basis may comprise hydrocarbons such as hard, soft or liquid paraffin, glycerol, beeswax, wax, a metallic soap; a mucilage; an oil of natural origin such as almond, corn, arachis, castor or olive oil; wool fat or its derivatives, or a fatty acid such as stearic or oleic acid together with an alcohol such as propylene glycol. The formulation may incorporate any suitable surface-active agent such as an anionic, cationic or non-ionic surfactant such as esters or polyoxyethylene derivatives thereof. Suspending agents such as natural gums, cellulose derivatives or inorganic materials such as silicaceous silicas, and other ingredients such as lanolin, may also be included.

Oral doses of the compounds of the invention in a pharmaceutical dosage unit will be an efficacious, nontoxic quantity preferably selected from the range of 0.001-100 mg/kg of active compound, preferably 0.001-50 mg/kg. When treating a human patient in need of treatment for steroid responsive dermatoses, the selected dose is administered preferably from 1-6 times daily. Oral dosage units for human administration suitably contain from 0.05 to 350 mg, suitably from 0.1 to 300 mg, suitably from 5 to 250 mg of active compound.

Optimal dosages to be administered may be readily determined by those skilled in the art, and will vary with the particular compound in use, the strength of the preparation, the mode of administration, and the advancement of the disease condition. Additional factors depending on the particular patient being treated will result in a need to adjust dosages, including patient age, weight, diet, and time of administration.

The method of this invention of treating steroid responsive dermatoses in mammals, including humans, comprises administering to a subject in need thereof a therapeutically effective amount of Compound A or a pharmaceutically acceptable salt thereof, Compound B, or Compound C.

The present invention relates to the use of Compound A or a pharmaceutically acceptable salt thereof, Compound B, or Compound C, in the treatment of steroid responsive dermatoses in a mammal, including a human.

The present invention relates to the in vivo administration of Compound A or a pharmaceutically acceptable salt thereof, Compound B, or Compound C, in the treatment of steroid responsive dermatoses in a mammal, including a human.

The invention also provides for the use of Compound A or a pharmaceutically acceptable salt thereof, Compound B, or Compound C, in the manufacture of a medicament for use in the treatment of steroid responsive dermatoses in mammals including humans.

The invention also provides for the use of Compound A or a pharmaceutically acceptable salt thereof, Compound B, or Compound C, in the manufacture of a medicament for use in therapy.

The invention also provides for a pharmaceutical composition for use in the treatment of steroid responsive dermatoses which comprises Compound A or a pharmaceutically acceptable salt thereof, Compound B, or Compound C, and a pharmaceutically acceptable carrier.

In addition, the pharmaceutically active compounds of the present invention can be co-administered with further active ingredients, such as other compounds known to treat steroid responsive dermatoses.

Without further elaboration, it is believed that one skilled in the art can, using the preceding description, utilize the present invention to its fullest extent. The following Examples are, therefore, to be construed as merely illustrative and not a limitation of the scope of the present invention in any way.

### EXPERIMENTAL DETAILS

### Example 1

### Capsule Composition

An oral dosage form for administering the present invention is produced by filing a standard two piece hard gelatin capsule with the ingredients in the proportions shown in Table 3, below.

TABLE 3

| INGREDIENTS | AMOUNTS |
|---|---|
| N-{(1S)-2-Amino-1-[(3-fluorophenyl)methyl]ethyl}-5- | 25 mg |
| chloro-4-(4-chloro-1-methyl-1H-pyrazol-5-yl)-2- | |
| thiophenecarboxamide hydrochloride | |
| Mannitol | 55 mg |
| Talc | 16 mg |
| Magnesium Stearate | 4 mg |

### Example 2

### Injectable Parenteral Composition

An injectable form for administering the present invention is produced by stirring 1.5% by weight of: N-{(1S)-2-amino-1-[(3,4-difluorophenyl)methyl]ethyl}-5-chloro-4-(4-chloro-1-methyl-1H-pyrazol-5-yl)-2-furancarboxamide in 10% by volume propylene glycol in water.

### Example 3

### Tablet Composition

The sucrose, microcrystalline cellulose and a compound of the invention, as shown in Table 4 below, are mixed and granulated in the proportions shown with a 10% gelatin solution. The wet granules are screened, dried, mixed with the starch, talc and stearic acid, then screened and compressed into a tablet.

TABLE 4

| INGREDIENTS | AMOUNTS |
|---|---|
| N-{(1S)-2-Amino-1-[(3-fluorophenyl)methyl]ethyl}-5- | 20 mg |
| chloro-4-(4-chloro-1-methyl-1H-pyrazol-5-yl)-2- | |
| thiophenecarboxamide hydrochloride | |
| Microcrystalline cellulose | 30 mg |
| sucrose | 4 mg |
| starch | 2 mg |
| talc | 1 mg |
| stearic acid | 0.5 mg |

### Example 4

### Topical Composition #1

Stearyl alcohol (60 grams) is heated to 80 C. USP olive oil (940 grams) is heated to the same temperature. While at 80 C., the stearyl alcohol is added to the preheated olive oil. 20 grams glycerin, 20 grams tri-stearin, 1 gram of an antioxidant mixture are added by agitation. 1 gram of N-{(1S)-2-Amino-1-[(3-fluorophenyl)methyl]ethyl}-5-chloro-4-(4-chloro-1-methyl-1H-pyrazol-5-yl)-2-thiophenecarboxamide is added and the mixture poured into containers (25 gram tubes) and allowed to cool spontaneously. While the mixture cooled to ambient temperature it gradually turns into a semi-solid.

### Example 5

### Topical Composition #2

Behenyl alcohol (10 grams) is heated to 80 C. Light paraffin oil (90 grams) is heated to the same temperature. While at 80 C., the behenyl alcohol is added to the preheated oil. One gram of N-{(1S)-2-Amino-1-[(3-fluorophenyl)methyl]ethyl}-5-chloro-4-(4-chloro-1-methyl-1H-pyrazol-5-yl)-2-thiophenecarboxamide hydrochloride is added and the mixture poured into containers (5 gram tubes) and allowed to cool spontaneously. While the mixture cooled to ambient temperature it gradually turns into a semi-solid.

### Example 6

### Topical Composition #3

Behenic acid (10 grams) is heated to 80 C. Light paraffin oil (90 grams) is heated to the same temperature. While at 80 C., the behenic acid is added to the preheated oil. Ten grams glycerin, 10 grams tristearin and 1 gram of an antioxidant mixture are added by agitation. 1 gram of N-{(1S)-2-amino-1-[(3,4-difluorophenyl)methyl]ethyl}-5-chloro-4-(4-chloro-1-methyl-1H-pyrazol-5-yl)-2-furancarboxamide is added and the mixture poured into containers (5 gram tubes) and allowed to cool spontaneously. While the mixture cooled to ambient temperature it gradually turns into a semi-solid.

### Example 7

### Topical Composition #4

12-hydroxy stearic acid (10 grams) is heated to 80 C. Light paraffin oil (90 grams) is heated to the same temperature. While at 80 C., the 12-hydroxy stearic acid is added to the preheated oil. Ten grams glycerin, 10 grams tri-stearin and 1 gram of an antioxidant mixture are added by agitation. 2.4 grams of N-{(1S)-2-Amino-1-[(3-fluorophenyl)methyl]ethyl}-5-chloro-4-(4-chloro-1-methyl-1H-pyrazol-5-yl)-2-thiophenecarboxamide hydrochloride are added and the mixture poured into containers (10 gram tubes) and allowed to cool spontaneously. While the mixture cooled to ambient temperature it gradually turns into a semi-solid.

While the preferred embodiments of the invention are illustrated by the above, it is to be understood that the invention is not limited to the precise instructions herein disclosed and that the right to all modifications coming within the scope of the following claims is reserved.

1. A method of treating steroid responsive dermatoses in a mammal in need thereof which comprises administering a therapeutically effective amount of a compound selected from: N-{(1S)-2-Amino-1-[(3-fluorophenyl)methyl]ethyl}-5-chloro-4-(4-chloro-1-methyl-1H-pyrazol-5-yl)-2-thiophenecarboxamide or a pharmaceutically acceptable salt thereof; and N-{(1S)-2-amino-1-[(3,4-difluorophenyl)methyl]ethyl}-5-chloro-4-(4-chloro-1-methyl-1H-pyrazol-5-yl)-2-furancarboxamide or a pharmaceutically acceptable salt thereof; to such mammal.

2. The method of claim 1 wherein the mammal is a human.

3. The method of claim 2 wherein the administered compound is N-{(1S)-2-Amino-1-[(3-fluorophenyl)methyl]ethyl}-5-chloro-4-(4-chloro-1-methyl-1H-pyrazol-5-yl)-2-thiophenecarboxamide or a pharmaceutically acceptable salt thereof.

4. The method of claim 2 wherein the administered compound is N-{(1S)-2-Amino-1-[(3-fluorophenyl)methyl]ethyl}-5-chloro-4-(4-chloro-1-methyl-1H-pyrazol-5-yl)-2-thiophenecarboxamide hydrochloride.

5. The method of claim 2 wherein the administered compound is N-{(1S)-2-Amino-1-[(3-fluorophenyl)methyl]ethyl}-5-chloro-4-(4-chloro-1-methyl-1H-pyrazol-5-yl)-2-thiophenecarboxamide.

6. The method claim 2 wherein the administered compound is N-{(1S)-2-amino-1-[(3,4-difluorophenyl)methyl]ethyl}-5-chloro-4-(4-chloro-1-methyl-1H-pyrazol-5-yl)-2-furancarboxamide or a pharmaceutically acceptable salt thereof.

7. The method of claim 1 wherein the steroid responsive dermatoses is selected from: contact dermatitis, eczema, infantile eczema, atopic dermatitis, ichthyosis, psoriasis, xeroderma, seborrheic dermatitis, nummular dermatitis, dermatitis herpetiformis, neurodermatitis, stasis dermatitis, lichen simplex chronicus, dermatophytids, candidiasis, intertrigo, scabies, pityriasis rosea, lichen planus, pityriasis rubra pilaris, bullous pemphigoid, miliaria, acute and chronic eczema, lupus erythematosis, photoallergic reactions, pruritis, and combinations thereof.

8. The method of claim 7 wherein the steroid responsive dermatoses is psoriasis.

9. The method of claim 7 wherein the compound is administered orally.

10. The method of claim 7 wherein the compound is administered topically.

11. The method of claim 7 wherein the mammal is a human.

12-16. (canceled)

17. The method of claim 10 wherein the mammal is a human.

18. A pharmaceutical composition suitable for topical administration comprising N-{(1S)-2-Amino-1-[(3-fluorophenyl)methyl]ethyl}-5-chloro-4-(4-chloro-1-methyl-1H-pyrazol-5-yl)-2-thiophenecarboxamide or a pharmaceutically acceptable salt thereof.

19-23. (canceled)

24. A pharmaceutical composition suitable for topical administration comprising N-{(1S)-2-amino-1-[(3,4-difluorophenyl)methyl]ethyl}-5-chloro-4-(4-chloro-1-methyl-1H-pyrazol-5-yl)-2-furancarboxamide or a pharmaceutically acceptable salt thereof.

25. The method claim 2 wherein the administered compound is N-{(1S)-2-amino-1-[(3,4-difluorophenyl)methyl]ethyl}-5-chloro-4-(4-chloro-1-methyl-1H-pyrazol-5-yl)-2-furancarboxamide.

26. (canceled)
